(19) United States
(12) Plant Patent Application Publication
(10) Pub. No.: US 2014/0317798 P1
(43) Pub. Date: Oct. 23, 2014

(54) Raspberry plant variety named enrosadira

(50) ;

(71) Applicant: AZ. AGR. Molari & Gatti Di Molari Gilberto, Cesena (IT)

(72) Inventor: Aldo Telch, Faver (IT)

(73) Assignee: AZ. AGR. Molari & Gatti Di Molari Gilberto, Cesena (IT)

(21) Appl. No.: 14/120,026

(22) Filed: Apr. 16, 2014

(30) Foreign Application Priority Data
Apr. 17, 2013 (IT) 20131117

(51) Int. Cl. A01H 5/00

(52) U.S. Cl. PLT/204

(57) ABSTRACT

The red raspberry cultivar “Enrosadira” was found in Trentino (Italy). The new cultivar is a primocane fruit-bearing variety producing two crops per year, wherein the fruits ripen on current season's cane in the middle of July and on previous season's cane in the beginning of June. The resulting fruits are conical and light red with a very good taste.

### BACKGROUND OF THE NEW VARIETY

The present invention concerns a new and distinct cultivar of red raspberry, botanical taxon Rubus idaeus L., hereinafter referred to by the cultivar name “Enrosadira”.

The new cultivar “Enrosadira” was discovered in 2004 in Trentino, Italy and has been asexually reproduced by root cutting since 2008. The new cultivar was found to be stable and its distinctive characteristics have been transmitted without change through succeeding (more than 98%) asexual propagations.

### SUMMARY OF THE INVENTION

The most outstanding and distinguishing characteristics of the new “Enrosadira” cultivar are briefly reported hereunder:
primocane fruit-bearing raspberry plant;
brilliant light red fruit colour and good flavour;
large, conical, and firm berries;
fruit having a very good shelf-life;
tolerance to raspberry pests and diseases.

The new cultivar can be propagated by the use of suckers produced in a spawn-bed, root cutting, and use of tissue culture techniques. Propagation by root cutting is preferred.

### BRIEF DESCRIPTION OF THE DRAWINGS

The new red raspberry cultivar “Enrosadira” is illustrated by the accompanying drawings, which are color photographs showing typical specimens of the new variety. The photographs are of “Enrosadira” raspberry plants that are two years old.

FIG. 1 is a color photograph of an “Enrosadira” raspberry plant of the present invention in a planted row showing the plant's general characteristics, including a typical distribution of fruits and leaf morphology.

FIG. 2 is a color photograph of an “Enrosadira” raspberry plant of the present invention showing both the ripe and immature fruit size, color, and shape.

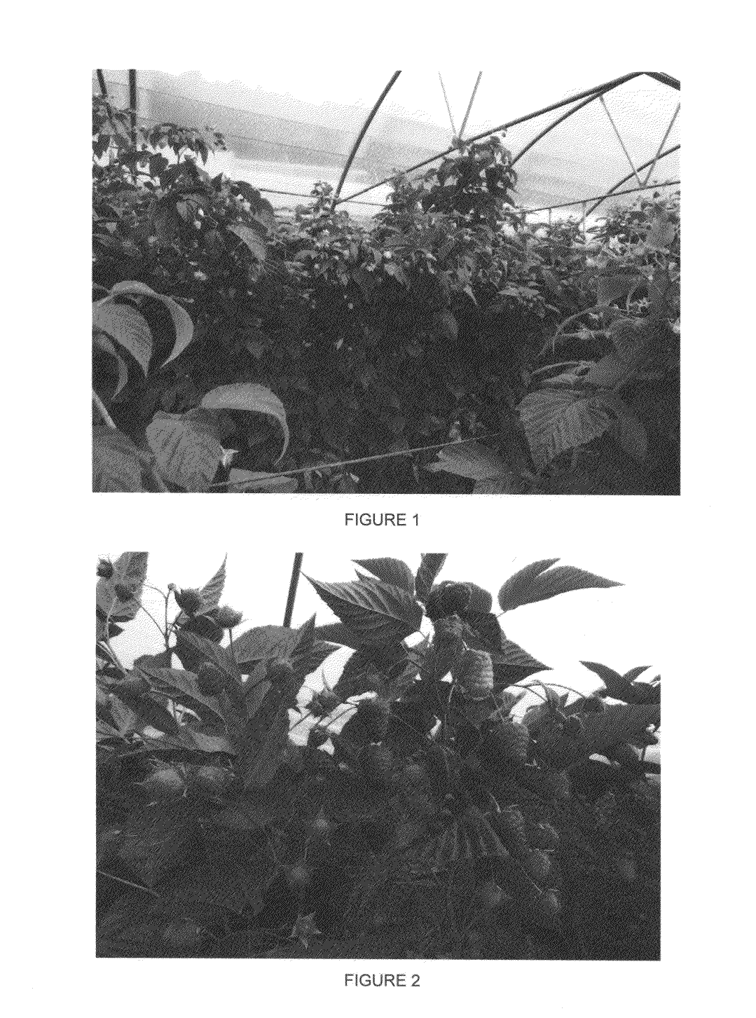

### DETAILED DESCRIPTION OF THE NEW CULTIVAR

The following detailed description sets forth the distinctive characteristics of the new raspberry variety “Enrosadira”.

The performance of the new cultivar for retention of its distinctive characteristics was evaluated through successive asexual propagations by root cutting in Trentino, Italy under conditions of temperature ranging from −8° C. in winter in an open greenhouse to 32° C. in summer in a closed greenhouse. The plants were grown in a peat substrate as soil.

A plant assessment was performed for four years.

The “Enrosadira” is a very early primocane fruit-bearing variety with fruit production beginning in early summer (June in Northern Italy) and lasting until autumn (September in Northern Italy). In autumn, the fruit production is small compared to early summer.
Classification:
   Family.—Rosaceae.
   Botanical taxon.—Rubus idaeus L.
   Common name.—Rasperry.
Plant:
   General characteristics.—upright canes, very vigorous, adapted to the medium-high winter chill conditions of Northern Italy; very good root system;
   Number of current season's shoots.—many to very many.
   Very young shoot.—anthocyanin coloration at apex during rapid growth is absent.
   Dormant cane colour.—brown.
   Spines.—present; thick and dense.
   Floral bloom.—simple with short laterals; the blooming begins in summer on the current year's shoots and in the spring season on the previous year's canes.
Fruit:
   General shape.—conical.
   Weight.—6-8 g.
   Colour of ripe fruit.—brilliant light red.
   Main bearing time.—both on previous year's cane in summer and on current year's cane in autumn.
   Time of beginning the fruit ripening on previous year's cane.—very early to early (middle of June in Northern Italy).
   Time of beginning the fruit ripening on current season's cane.—very early (middle of July in Northern Italy).
   Storage.—very good shelf life in conditioned storage, yields good quality fruits.
Pest and disease resistance: The new variety “Enrosadira” is resistant to Aphid ruby, Phytophthora c., and Didymella appl. The new cultivar “Enrosadira” is similar to, but distinct from, the commercial variety “Erika” (U.S. Plant Pat. No. 20,841) as shown in Table 1.

TABLE 1
Comparison between “Enrosadira” and “Erika”

| Characteristic | “Enrosadira” | “Erika” |
|---|---|---|
| Plant | Medium-high, vigorous | Medium |
| Dormant cane color | Brown | Brown |
| Spines | Thick and dense | Thick and dense |
| Fruit | Brilliant light red | Light red |
| | Conical | Broad conical |
| Fruit ripening time on the | Middle of June in | Beginning of July in |
| previous season's cane | Northern Italy | Northern Italy |

What is claimed:

1. A new and distinct cultivar of red raspberry plant named “Enrosadira”substantially as herein shown and described.
